(19) United States
(12) Patent Application Publication
(10) Pub. No.: US 2008/0239476 A1
(43) Pub. Date: Oct. 2, 2008

(54) Illuminating module and surgical microscope incorporating said illuminating module

(76) Inventors: Holger Matz, Unterschneidheim (DE); Bryce Anton Moffat, Jena (DE)

Correspondence Address: WALTER OTTESEN, PO BOX 4026, GAITHERSBURG, MD, 20885-4026, US

(21) Appl. No.: 12/076,135

(22) Filed: Mar. 14, 2008

(30) Foreign Application Priority Data
Mar. 14, 2007 (DE) 10 2007 012 951.5

(51) Int. Cl. F21V 13/00; A61B 19/00; G02B 21/06

(52) U.S. Cl. 359/389; 362/231

(57) ABSTRACT

An illuminating module (100) has a light source which has at least one white-light LED. In addition to the white-light LED, an LED for red light and an LED for green light are provided. The illuminating module (100) has an output (112) and includes a light-mixing unit (101) which mixes the light of the white-light LED with the light of the respective LEDs for red light and green light in order to make available white illuminating light at the output (112) of the illuminating module for illuminating light.

### CROSS REFERENCE TO RELATED APPLICATION

This application claims priority of German patent application no. 10 2007 012 951.5, filed Mar. 14, 2007, the entire content of which is incorporated herein by reference.

### FIELD OF THE INVENTION

The invention relates to an illuminating module having a light source which has at least one white-light LED.

### BACKGROUND OF THE INVENTION

In a surgical microscope, in order to make the area of surgery bright and visible with good contrast for a surgeon, the area of surgery must be illuminated with a high intensity and true color light source.

It is known to utilize halogen lamps and xenon lamps as light sources. Such light sources operate as thermal radiators. A halogen lamp generates light having a spectral intensity which corresponds approximately to that of a black radiator at a temperature T in the range T=2800° K to T=3200° K. A xenon lamp emits light having a spectral intensity which corresponds approximately to that of a black radiator of the temperature T=4800° K.

Xenon lamps or halogen lamps as light sources have the disadvantage as thermal radiators that the light generation is associated with an intense development of temperature. The service life of these lamps is therefore limited and the spectral intensity of the light emitted by these lamps is not constant over the service life thereof.

Illuminating modules, which generate light via LEDs, do not have this disadvantage. LEDs generate light at a comparatively low electric power without generating as much heat as thermal radiators. LEDs can be produced more cost effectively and can be operated over a longer service life compared to xenon lamps and halogen lamps.

In LEDs, light is generated by electroluminescence which arises in that, in the luminescent diodes, charge carriers transfer from one quantum state into another quantum state and, in doing so, output energy which is converted into light. Wavelength spectrums of light, which is generated by electroluminescence, is therefore of a narrow bandwidth compared to a thermal radiator.

To generate white light with LEDs, it is known to superpose the light from three or more differently-colored luminescent diodes of the colors red, green and blue one upon the other or to convert the narrow band light, which is generated by electroluminescence, to white light, for example, in that phosphor-containing conversion material is transilluminated with light of a blue LED. When the lights of LEDs of different colors are mixed to white light, then the color temperature of the resulting white light can be adjusted by varying the light intensity emitted by the individual LEDs. The color temperature of a light source is understood to be that color temperature of a black radiator which imparts to the black radiator the color impression of the light source.

Illuminating modules which generate white light by mixing the light of luminescent diodes of the colors red, green and blue or wherein white light is generated in that the light of a blue LED is passed through phosphor-containing conversion material, can generate only white light whose so-called CRI-value is low compared to that of white light from a halogen light source or xenon light source.

In the following, it is understood that the CRI-value of the white light, which is emitted by the illuminating module, is the numerical value which is given in the formula 19.9 on page 317 of the text of E. Fred Schubert entitled “Light Emitting Diodes”, Cambridge University Press, Second Edition 2006. This numerical value is, with reference to a reference light source, an index for the color fidelity of a standardized color table illuminated by the illuminating module as a light source. In the following, the CRI-value is referred to a thermal black body radiator of the temperature T=4800° K.

For illuminating modules, which are known from the state of the art and which generate white light by superposing the light from a red (R) LED, green (G) LED and blue (B) LED, the following applies:

CRI_{LED-Light source}<80

### SUMMARY OF THE INVENTION

It is an object of the invention to provide a surgical microscope illuminating module wherein light is generated with LEDs and wherein white illuminating light is made available with variable color temperature at simultaneously maximum CRI-value where the following relationship applies:

CRI_{Illuminating module}>80

The above object is realized with an illuminating module of the kind described above wherein an LED for red light (R) and an LED for green light (G) are provided. The illuminating module includes a light-mixing unit which mixes the light of the white-light LED with light of the LED for red light (R) and the light of the LED for green light (G) in order to make available white illuminating light at an output of the illuminating module.

In this way, an illuminating module is provided which makes white light available. The color reproduction of this white light compared to the white light, which is generated with white-light LEDs or with RGB-LEDs, is improved so that a natural viewing impression is made possible for an observer over a wide color range.

According to another feature of the invention, a control unit is provided to control the light intensity outputted by each LED. In this way, it is possible to adjust the spectral intensity of the illuminating light outputted by the illuminating module.

According to another feature of the invention, a data store is assigned to the control unit which, for the different color temperatures, contains the required currents of the respective LEDs for white light, red light and green light in order to generate white light of the particular color temperature at the maximum CRI-value with the illuminating module. In this way, the color reproduction can be optimized with the illuminating module.

According to another feature of the invention, the illuminating module includes an input unit for inputting a desired color temperature of the white light emitted by the illuminating module. In this way, the color temperature of the white light, which is generated with the illuminating module, can be adapted to the requirements of an observed object region.

### BRIEF DESCRIPTION OF THE DRAWINGS

The invention will now be described with reference to the drawings wherein:

FIG. 1 is a schematic of the illuminating module according to the invention;

FIG. 2 is a perspective view of a chip-mixing module having two white-light LEDs and a red-light (R) LED as well as a green-light (G) LED;

FIG. 3 is a schematic showing the configuration of a white-light LED in the chip-mixing module;

FIG. 4 is a schematic showing the configuration of a red-light (R) LED or a green-light (G) LED in the chip-mixing module;

FIG. 5 is a graph showing the spectral intensity of the white light emitted by the illuminating module for differently selected color temperatures;

FIG. 6 is a detail of a the color triangle in accordance with the CIE standard with color values for light from the chip-mixing module;

FIG. 7 is a graph showing CRI-values for white light from the chip-mixing module which are referred to a thermal black body radiator of the temperature T=4800° K; and,

FIG. 8 is a schematic of a surgical microscope having an illuminating unit in the form of the illuminating module.

### DESCRIPTION OF THE PREFERRED EMBODIMENTS OF THE INVENTION

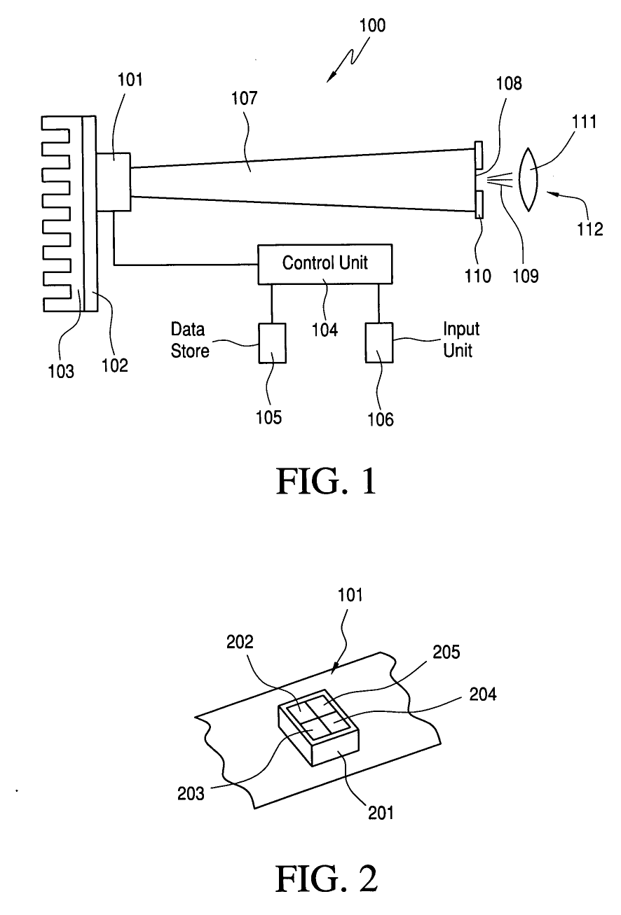

The illuminating module 100 of FIG. 1 contains a chip-mixing module 101 which is mounted on a carrier base 102. The carrier base 102 is connected to a cooling unit 103. In the chip-mixing module 101, the following are arranged as light source: two white-light LEDs, a red-light (R) LED and a green-light (G) LED.

The chip-mixing module 101 is connected to a control unit 104. A data store 105 and an input unit 106 are assigned to the control unit 104. The control unit 104 controls the current flow through the two white-light LEDs and the red-light LED (R) and the green-light LED (G) in the chip-mixing module 101.

In the illuminating module 100, the light emanating from the chip-mixing module 101 passes through an integrator rod 107. The integrator rod 107 functions as a light-mixing unit and homogenizes the light emitted by the chip-mixing module. The integrator rod 107 has an output end 108 whereat white light 109 exits from the integrator rod 107. At its outlet end 108, the integrator rod 107 is closed off by an illuminating field diaphragm 110. A lens 111 having a positive refractive power is assigned to the illuminating field diaphragm 110. This lens 111 has a positive refractive power and images the illuminating field diaphragm 110 at infinity so that a parallel illuminating light beam is made available at the output 112 of the illuminating module 100.

FIG. 2 shows a perspective view of the chip-mixing module 101 of FIG. 1. The chip-mixing module 101 has a holder 201 wherein the following are accommodated: a first white-light LED 202, a second white-light LED 203, a red-light (R) LED 204 and a green-light (G) LED 205.

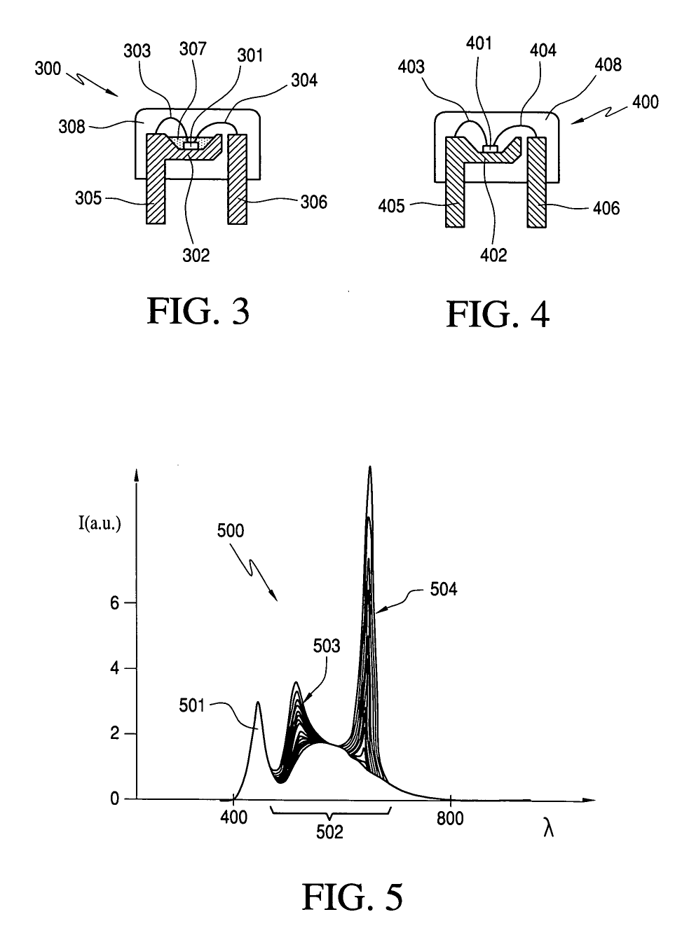

The schematic configuration of the white-light LEDs 202 and 203 in the chip-mixing module 101 is shown in FIG. 3 and described hereinafter.

The white-light LED 300 contains an LED chip 301 comprising GaInN/GaN. This chip is mounted on a carrier body 302 and is connected via contact leads 303 and 304 to first and second electric connections (305, 306), respectively. The LED chip 301 is embedded in a phosphor layer 307 in the carrier body 302. The operating principle of the white-light LED is described on page 353 of the text by E. Fred Schubert entitled “Light Emitting Diodes”, Cambridge University Press, Second Edition 2006, and incorporated herein by reference. The LED chip 301 emits blue light which generates yellow light when passing through the phosphor layer 307. This yellow light forms white light when superposed with the blue LED light as an additive spectral color mixture. The white light generated in this manner is then outputted to the ambient via a plastic body 308 in which the arrangement is cast.

FIG. 4 shows the configuration of the red-light (R) LED or the green-light (G) LED in the chip-mixing module 101 of FIG. 1. The corresponding LED contains an LED chip 401. For generating red light, this LED chip comprises an AlGaAs/GaAs-heterostructure. Green light can be generated with an LED chip of a GaAsP:N-heterostructure. As in the case of the white-light LED, the LED chip 401 is arranged in a carrier body 402 and is connected to first and second electrical connections (405, 406) via contact leads (403, 404), respectively. The light emitted by the LED chip is outputted to the ambient via a plastic jacket 408.

It is also possible to configure the chip-mixing module with white-light, red-light and green-light LEDs which are placed on a common carrier substrate in the chip-mixing module. Preferably, the LEDs with the carrier substrate are jacketed by a common plastic body.

In FIG. 5, the curve 500 shows the spectral intensity I of the illuminating light, which is outputted by the illuminating module 100, for different currents. The currents are shown in arbitrary units (a.u.) and are conducted through the LEDs in the mixing module. A base brightness of the white light, which is outputted by the illuminating module, is achieved with the two white-light LEDs in the mixing module 101 of FIG. 1. The course of the spectral intensity of the light, which is emitted by the white-light LEDs, is characterized by a local maximum 501 in the blue spectral range. This maximum is based on the situation that in the white-light LED, the white light is generated by conversion from blue light which passes through a phosphor layer and transforms the generated blue light into the wavelength range 502 of FIG. 5.

The white-light luminescent diodes in the chip-mixing module 101 of FIG. 1 are preferably driven for maximum light intensity.

By adjusting the current, which is supplied to the red-light (R) LED and the green-light (G) LED, the intensity of the illuminating light, which is outputted by these luminescent diodes, can be adjusted in order to adjust the peaks of the local maxima 503 and 504 in the spectral intensity of the illuminating light emitted by the illuminating module 100 of FIG. 1. In this way, the color temperature of the illuminating light and the corresponding CRI-value of this light can be varied.

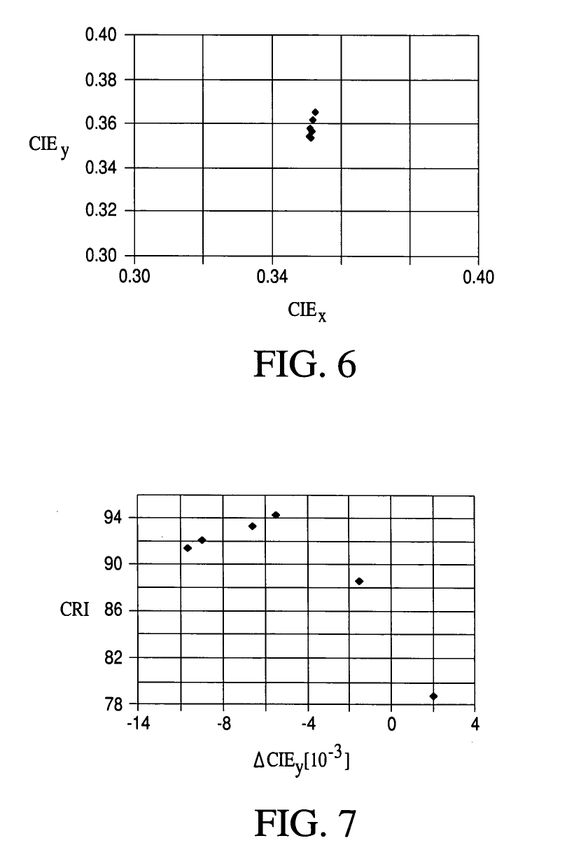

FIG. 6 shows a part of the color triangle according to the CIE standard shown on page 308 in the text by E. Fred Schubert entitled “Light Emitting Diodes”, Cambridge University Press, Second Edition 2006 which is incorporated herein by reference. In this color triangle, for 6 different values of the integral relative intensity I_{W}, I_{R} and I_{G}, the corresponding CIE-value coordinates (CIEₓ; CIE_{y}) for the white light are shown which white light is generated by the white, red and green LEDs in the chip-mixing module 101 of FIG. 1.

The color temperature T_{Color} of the generated white light lies in the range between 4785° K<T_{Color}<4811° K. The values for the integral relative intensity of the illuminating light outputted by the LEDs in the chip-mixing module form the basis for the CIE-value coordinates (CIEₓ; CIE_{y}) as shown in the following table.

TABLE

| I_{R}:I_{W}:I_{G} | T_{Color}° K | (CIEₓ; CIE_{y}) | CRI |
|---|---|---|---|
| 1.34:7.40:1.05 | 4798 | (0.3519; 0.3634) | 78.9 |
| 1.63:13.01:1.00 | 4785 | (0.3508; 0.3537) | 91.4 |
| 1.63:11.67:1.09 | 4787 | (0.3511; 0.3568) | 93.3 |
| 1.54:9.47:1.15 | 4799 | (0.3514; 0.3619) | 89.3 |
| 1.57:12.54:1.00 | 4802 | (0.3504; 0.3543) | 92.1 |
| 1.64:11.68:1.15 | 4811 | (0.3506; 0.3579) | 94.3 |

It can be seen that for comparatively insignificant changes of the color temperature T_{Color} of the light, which is emitted by the chip-module 101, clear variations of the resulting CRI-value for the generated light result.

The CRI-value of the white light, which is generated by the LEDs in the chip-mixing module 101, is shown for different values of the corresponding relative integral intensities I_{R}, I_{W} and I_{G} in FIG. 7. There, the CRI-value of the corresponding generated white light is plotted against the deviation ΔCIE_{y} of the value of CIE_{y} from the CIE color coordinates (CIEₓ; CIE_{y}) to the CIE_{y}-value of the black temperature radiator at T=4800° K. The CRI-value can be adjusted by varying the relative intensity of the LEDs in the chip-mixing module 101. According to the above, for this CRI-value, a maximum above CRI=94 can be adjusted. With the corresponding adjustment of the relative integral intensity of the LEDs in the chip-mixing module, white light can be generated for optimal color reproduction. In this way, CRI-values>80 can be adjusted for light from the illuminating module 100 of FIG. 1.

For a pregiven color temperature, those currents for the white-light LED and the red-light LED and green-light LED in the mixing module 101 of the illuminating module are stored in the data store 105 of the illuminating module 100 of FIG. 1 for which the CRI-value is a maximum. A desired color temperature can be selected via the input unit 106 in the illuminating module 100. For this selected color temperature, the control unit 104 uses the appropriate currents for the LEDs in the mixing module 101 in order to generate illuminating light having a maximum CRI-value.

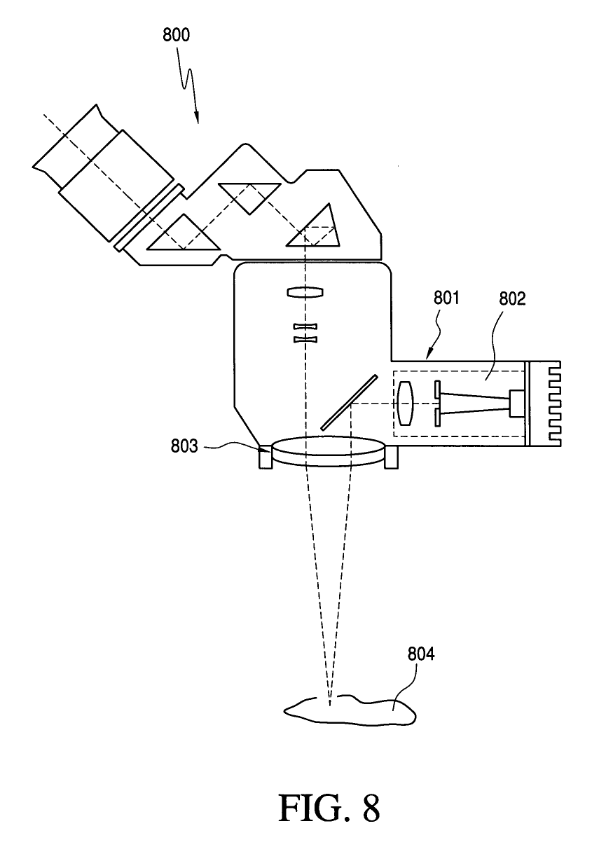

FIG. 8 shows a surgical microscope 800 which contains an illuminating unit 801 having an illuminating module 802 which has the configuration explained with respect to FIG. 1. The illuminating unit generates illuminating light which is directed via the microscope main objective 803 to the object region 804 of the surgical microscope in order to illuminate the same.

It is understood that the foregoing description is that of the preferred embodiments of the invention and that various changes and modifications may be made thereto without departing from the spirit and scope of the invention as defined in the appended claims.

What is claimed is:

1. An illuminating module for providing illuminating light, the illuminating module having an output and comprising:
a light source including a white-light LED emitting light, a red-light LED emitting light and a green-light LED emitting light; and,
a light-mixing unit for mixing said light of said white-light LED with said light of said red-light LED and said light of said green-light LED in order to provide white illuminating light at said output of said illuminating module.

2. The illuminating module of claim 1, further comprising a control unit for controlling the light intensity outputted by each of said LEDs.

3. The illuminating module of claim 2, further comprising a data store operatively connected to said control unit; and, said data unit containing required respective currents of said LEDs for said white light, said red light and said green light to generate, with said illuminating module, white light of a particular color temperature at a maximum CRI-value.

4. The illuminating module of claim 3, further comprising an input unit for inputting a wanted color temperature of said white light outputted by said illuminating module.

5. A surgical microscope for viewing a region of an object, said surgical microscope comprising an illuminating module for providing illuminating light, said illuminating module having an output and including:
a light source including a white-light LED emitting light, a red-light LED emitting light and a green-light LED emitting light; and,
a light-mixing unit for mixing said light of said white-light LED with said light of said red-light LED and said light of said green-light LED in order to provide white illuminating light at said output of said illuminating module.

6. The surgical microscope of claim 5, wherein said illuminating module further comprises a control unit for controlling the light intensity outputted by each of said LEDs.

7. The surgical microscope of claim 6, wherein said illuminating module further comprises a data store operatively connected to said control unit; and, said data unit containing required respective currents of said LEDs for said white light, said red light and said green light to generate, with said illuminating module, white light of a particular color temperature at a maximum CRI-value.

8. The surgical microscope of claim 7, wherein said illuminating module further comprises an input unit for inputting a wanted color temperature of said white light outputted by said illuminating module.
